(19) United States
(12) Patent Application Publication
(10) Pub. No.: US 2018/0137935 A1
(43) Pub. Date: May 17, 2018

(54) EDIBLE RECOMMENDATION

(71) Applicant: KONINKLIJKE PHILIPS N.V., EINDHOVEN (NL)

(72) Inventors: Johannes Henricus Maria Korst, Eindhoven (NL); Serverius Petrus Paulus Pronk, Vught (NL); Mauro Barbieri, Eindhoven (NL); Marc Andre Peters, Veldhoven (NL); Henriette Christine Marie Hoonhout, Eindhoven (NL); Ramon Antoine Wiro Clout, Eindhoven (NL)

(73) Assignee: KONINKLIJKE PHILIPS N.V.

(21) Appl. No.: 15/570,113

(22) PCT Filed: Apr. 12, 2016
(86) PCT No.: PCT/IB2016/052062

§ 371 Date: Oct. 27, 2017

Related U.S. Application Data
(60) Provisional application No. 62/155,825, filed on May 1, 2015.

(51) Int. Cl. G16H 20/60; A61B 5/00; G09B 19/00

(52) U.S. Cl. CPC G16H 20/60; G09B 19/0092; A61B 5/7275

(57) ABSTRACT

Systems and methods recommending edibles for consumption after considering a user's edible intake over a period of time. Each edible item, consumed or recommended, is associated with a set of parameters (or parameter ranges) that represent the various nutrients contained in that edible item (e.g., vitamins, minerals, calories, fat, etc.). The consumed items can be aggregated in various ways (simple averages, moving averages, weighted averages) to yield an overall indicator of the user's nutritional state and, given a goal nutritional state, edibles can be recommended for consumption that advance the user's nutritional state towards a goal nutritional state.

### FIELD

The present invention generally relates to improving health through food choices, and more specifically to the recommendation of edibles that are specifically tailored to dietary needs.

### BACKGROUND

Food recommendation has been an active area of research for a number of years. Previous attempts focused on learning a user's likes and dislikes of particular ingredients, cuisines, flavors, etc. By receiving feedback on previous recommendations, the recommender learns the user's preferences, sometimes maintaining a blacklist of ingredients that a user specifically dislikes.

More recent efforts have attempted to account for the healthiness of recommended meals. Meals can be recommended with the goal of solving dietary deficiencies or surpluses of particular nutrients instead of meals that may not address these nutritional issues.

However, a drawback of current efforts is that the recommendation of a food is typically made in isolation, not taking into account the long-term healthiness of an individual's eating habits. For example, the consistent recommendation of low fat recipes over high fat recipes may lead to nutritional imbalances, since unsaturated fats contain important fat-soluble nutrients. Moreover, a recommender that always offers healthy recommendations may be perceived as unattractive or rejected by users interacting with the recommender.

Accordingly, there is a need for methods and systems that recommend edibles for users that avoid these deficiencies.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

Embodiments of the present invention recommend edibles for consumption after considering a user's recent history of edible intake. Each edible item, consumed or recommended, is associated with a set of parameters (or parameter ranges) that represent the various nutrients contained in that edible item (e.g., vitamins, minerals, calories, fat, etc.). The consumed items can be aggregated in various ways (simple averages, moving averages, weighted averages) to yield an overall indicator of the user's nutritional state and, given a goal nutritional state, edibles can be recommended for consumption that advance the user's nutritional state towards the goal nutritional state.

In one aspect, embodiments of the present invention relate to a computer-implemented method for recommending edibles for consumption. The method includes providing a computer processor configured to maintain a historical profile of edible intake for an individual, compute a goal profile between the historical profile and a desired profile for the individual, and recommend at least one edible for consumption by the individual, wherein the recommended at least one edible has a nutrition profile that moves the historical profile closer to the goal profile, wherein each profile comprises a vector of parameters, with each parameter associated with a nutrient.

In one embodiment, the computer processor is further configured to receive an indication of at least one edible consumed by the individual, convert the indication of the at least one consumed edible into at least one edible consumed profile, and update the historical profile to incorporate the at least one edible consumed profile, wherein the edible consumed profile comprises a vector of parameters, with each parameter associated with a nutrient.

In one embodiment, updating the historical profile comprises computing a weighted average of the historical profile and the at least one edible consumed profile. In one embodiment, updating the historical profile comprises computing an unweighted average of the historical profile and the at least one edible consumed profile.

In one embodiment, each parameter of the goal profile is a range of values defined by a lower bound and an upper bound. In one embodiment, the historical profile comprises a plurality of vectors. In one embodiment, the recommendation applies different weights to different parameters. In one embodiment, recommending at least one edible for consumption comprises selecting at least one edible from a database of nutritional profiles for at least one edible.

In one embodiment, the goal profile is one of a plurality of intermediate goal profiles between the historical profile and the desired profile. In one embodiment, the goal profile is computed so as to be realizable by the individual in a reasonable time period. In one embodiment, the goal profile is the desired profile.

In one aspect, embodiments of the present invention relate to a computer readable medium containing computer-executable instructions for performing a method for recommending at least one edible for consumption. The medium comprises computer-executable instructions for maintaining a historical profile of edible intake for an individual, computer-executable instructions for communicating at least part of the historical profile to a processing module, the processing module configured to compute a goal profile between the historical profile and a desired profile for the individual, computer-executable instructions for receiving information from the processing module, and computer-executable instructions for recommending at least one edible for consumption by the individual, wherein the recommended at least one edible has a nutrition profile that moves the historical profile closer to the goal profile, wherein each profile comprises a vector of parameters, with each parameter associated with a nutrient.

In one embodiment, the computer readable medium further comprises computer-executable instructions for receiving an indication of at least one edible consumed by the individual, computer-executable instructions for converting the indication of at least one consumed edible into at least one edible consumed profile, and computer-executable instructions for updating the historical profile to incorporate the at least one edible consumed profile, wherein each edible consumed profile comprises a vector of parameters, with each parameter associated with a nutrient.

In one embodiment, updating the historical profile comprises computing a weighted average of the historical profile and the at least one edible consumed profile. In one embodiment, updating the historical profile comprises computing an unweighted average of the historical profile and the at least one edible consumed profile.

In one embodiment, the recommended edible for consumption is determined by the processing module and provided in the received information. In one embodiment, at least one parameter in the goal profile is a range of values defined by a lower bound and an upper bound. In one embodiment, the historical profile comprises a plurality of vectors. In one embodiment, the recommendation applies different weights to different parameters. In one embodiment, the computer-executable instructions for recommending at least one edible for consumption comprise computer-executable instructions for selecting at least one edible from a database of nutritional profiles for at least one edible.

In one embodiment, the goal profile is one of a plurality of goal profiles equidistant between the historical profile and the desired profile. In one embodiment, the goal profile is computed so as to be realizable by the individual in a reasonable time period. In one embodiment, the goal profile is the desired profile.

In one aspect, embodiments of the present invention relate to a computer readable medium containing computer-executable instructions for performing a method for recommending at least one edible for consumption. The medium comprises computer-executable instructions for maintaining a historical profile of edible intake for an individual, computer-executable instructions for computing a goal profile between the historical profile and a desired profile for the individual, and computer-executable instructions for recommending at least one edible for consumption by the individual, wherein the recommended at least one edible has a nutrition profile that moves the historical profile closer to the goal profile, wherein each profile comprises a vector of parameters, with each parameter associated with a nutrient.

These and other features and advantages, which characterize the present non-limiting embodiments, will be apparent from a reading of the following detailed description and a review of the associated drawings. It is to be understood that both the foregoing general description and the following detailed description are explanatory only and are not restrictive of the non-limiting embodiments as claimed.

### BRIEF DESCRIPTION OF DRAWINGS

Non-limiting and non-exhaustive embodiments are described with reference to the following figures in which:

FIG. 1 is an example of edible recommendations changing the nutritional content of a user's historical edible intake to the nutritional content of a desired goal state in accord with the present invention;

FIG. 2 depicts the use of a plurality of goal states to facilitate large changes in nutritional content in accord with the present invention;

FIG. 3 presents an exemplary embodiment of a method for edible recommendation in accord with the present invention; and

FIG. 4 is a schematic representation of an embodiment of an apparatus for edible recommendation according to the present invention.

In the drawings, like reference characters generally refer to corresponding parts throughout the different views. The drawings are not necessarily to scale, emphasis instead being placed on the principles and concepts of operation.

### DETAILED DESCRIPTION

Various embodiments are described more fully below with reference to the accompanying drawings, which form a part hereof, and which show specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are the means used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the present invention include process steps and instructions that could be embodied in software, firmware or hardware, and when embodied in software, could be downloaded to reside on and be operated from different platforms used by a variety of operating systems.

The present invention also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The processes and displays presented herein are not inherently related to any particular computer or other apparatus. Various general purpose systems may also be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein, and any references below to specific languages are provided for disclosure of enablement and best mode of the present invention.

In addition, the language used in the specification has been principally selected for readability and instructional purposes, and may not have been selected to delineate or circumscribe the inventive subject matter. Accordingly, the disclosure of the present invention is intended to be illustrative, but not limiting, of the scope of the invention, which is set forth in the claims.

Embodiments of the present invention concern the recommendation of edibles that are specifically tailored to the dietary needs of an individual. By representing relevant nutrients as separate dimensions of a multi-dimensional vector, the nutritional content of individual edibles as well as the long-term dietary needs of an individual may be represented as vectors. Specific edibles are recommended based on how well the edible, in combination with the vector(s) representing the individual's recent edible intake, approximates a goal vector representing the specific long-term dietary needs of the individual.

### Nutrient Vectors

To be able to propose edibles matched to a user's recent eating history, edibles (i.e., individual food items, recipes, meals, combinations of individual food items, etc.) are modeled in embodiments of the present invention as multi-dimensional vectors, with each dimension representing a specific nutrient that should be monitored to judge the healthiness of a user's eating habits. Exemplary macro-nutrients that can represented in this vector format include, but are not limited to, saturated fat, salt, carbohydrates, protein, etc. A vector may also include vitamins and minerals, either generalized or, in some embodiments, tailored to a particular person or a particular medical condition, such as folic acid for pregnant women or vitamin D for the elderly.

Still other embodiments include non-nutrient parameters in nutrient vectors, such as points or other rewards for consuming particular foods, although, for simplicity's sake, the following discussion focuses on nutrient vectors that only include nutrient parameters.

Once the parameters have been selected for the vector, the nutritional content for an edible is represented in the vector format, i.e., e=(e₁, e₂, . . . eₘ), where eᵢ is the contribution of the edible to the user's intake of the nutrient i.

The user's recent eating history h can be modelled as a profile of the cumulative nutritional contributions of individually-consumed edibles over a certain period, including suggested recipes, snacks, meals, etc. In various embodiments the historical profile h can be a vector h=(h₁, h₂, . . . hₘ) that is the sum of the nutritional content vectors for the preceding edibles, or it can be a historical record of the individual nutritional content vectors. While the former is sufficient for edible recommendations, as discussed below, a historical profile of individual vectors permits various additional features of interest, such as the computation of weighted averages, etc.

A desired nutritional state for a particular user can be modelled as a goal vector g over the relevant nutrients, i.e., g=(g₁, g₂, . . . gₘ), if for a nutrient i the desired intake for that nutrient can be expressed as a single value gᵢ. Given that the optimal intake for a particular nutrient often depends on a user's individual characteristics, e.g., medical condition, genetic predisposition, etc., then in some circumstances the optimal intake of a nutrient is better expressed as a range [lᵢ . . . uᵢ] where the values between lower bound lᵢ and upper bound uᵢ are considered healthy for the particular user. In that case, the goal of desired nutrient intake can be expressed as a hypercube g=([l₁,u₁],[l₂,u₂] . . . [lₘ,uₘ]); when the lower and upper bounds for one or more nutrients are identical, then the hypercube will be degenerate. In still other embodiments, the goal vector may be a combination of specific values for particular nutrients and desired ranges for other nutrients in the vector.

Let d(h, g) denote the “distance” between the vector representing the nutritional contributions of the eating history h of a user and the desired nutritional state g for that user as measured by a suitably chosen distance measure, possibly tailored to the user's individual condition. For example, some distance measures may be weighted, giving more weight to some nutrient dimensions at the expense of other nutrient dimensions.

Then, in accord with the present invention, an edible item with nutritional content r that results in a decreased “distance” between the nutritional contributions of the updated eating history profile u including the nutritional content of the new edible, i.e., u=c(h,r), where c( ) is representative of vector operations, such as a vector moving average, a smoothed vector average, etc., and the goal nutritional state g=(g₁, g₂, . . . gₘ) can be considered eligible for recommendation to the user. Among the set of eligible edible items (individual foods, recipes, meals, etc.), the ones for which d(u, g) is sufficiently small or, when d(h, g) is relatively large, the ones for which d(h, g)-d(u, g) is sufficiently large are considered eligible.

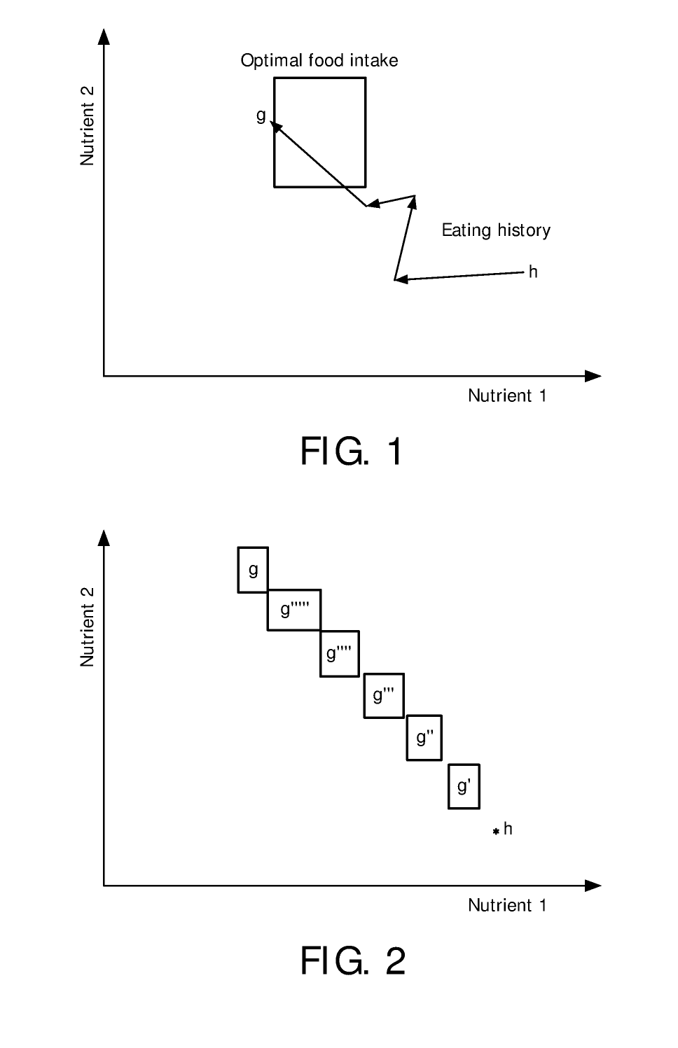

FIG. 1 presents a simple example of these concepts in graphical form. For simplicity, assume that only two nutrients are relevant to express edible nutritional content e, nutritional contributions from eating history profile h, and desired nutritional state g. FIG. 1 presents the desired nutritional state g as a shaded rectangle, i.e., a hypercube in two dimensions that represents desired ranges for the two nutrients at issue. The nutritional contributions from the user's eating history is identified by the starting point h.

The arrows indicate a possible trajectory from the nutritional contributions from the user's present eating history h towards the desired nutritional state g implemented as a series of edible item recommendations with each arrow representing the nutritional content of a recommended edible item.

As indicated, the recommendations consist of several individual edibles whose nutritional content is deficient in nutrient 1, but supply nutrient 2. Note that this situation may occur when the update rule c(h, r) is, e.g., a moving average. When a nutrient i has a history value hᵢ and recommended edible r has a corresponding nutritional value rᵢ, an update to h′ for this nutrient using a moving average would take the form h′ᵢ=αᵢ·rᵢ+(1−αᵢ)·hᵢ, with αᵢϵ(0, 1). The value of αᵢ may be selected depending on the important of the particular nutrient. Nutrients important to health may be associated with a higher α value; unimportant nutrients may be associated with a lower a value. Now, if rᵢ<hᵢ, then h′ᵢ<hᵢ. In other words, by not eating enough of a certain nutrient, its value is lowered.

In some cases, the distance between the nutritional content of the individual's historical profile of edible intake and the desired nutritional goal is too large, in the sense that the distance may exceed a threshold that is, e.g., predetermined or specific to the individual. One interpretation of such a condition is that the difference between the individual's present nutritional state, as exemplified by the individual's eating history, and the desired goal nutritional state is too large to be addressed by, for example, a single edible, a single meal, or edible consumption in the immediate future, etc. This may occur when the individual has developed bad eating habits over time and a strict diet over an intermediate time period (or longer) would be necessary to improve the individual's nutritional profile.

When the distance is too large, the recommendations required to reach the goal nutritional state may be so strict as to discourage the individual from proceeding with the program. In this case, one or more intermediate goal nutritional states between the nutritional content of the user's historical consumption and the desired goal nutritional state may be computed, and embodiments of the present invention may proceed to recommend at least one edible for consumption by the individual that reduces the distance between the individual's current nutritional state, as exemplified by the individual's historical profile of edible intake, and the computed intermediate goal nutritional state.

The intermediate goal nutritional state may be equidistant between the nutritional content of the user's historical profile and the desired nutritional state or, as illustrated in FIG. 2, one of a plurality of goal nutritional state (g′, g″, etc.) between the nutritional content of the user's historical profile h and the desired nutritional state g. The initial goal profile g′ may be an intermediate state between the nutritional content of the user's historical profile h and the desired nutritional state g that is sufficiently close to the nutritional content of the individual's current eating habits such that it can be reached by the individual in a reasonable time or a reasonable number of recommendations. Subsequent intermediate goal nutritional states g″, g′″, etc., consisting of small improvements may then form a trajectory between the first intermediate nutritional goal state and the desired nutritional state, each step from one intermediate nutritional goal state to the next being sufficiently close to each other so as to enable reaching the next intermediate nutritional goal state in a reasonable amount of time or a reasonable number of recommendations. As depicted in FIG. 2, each intermediate goal nutritional state in this embodiment is a hypercube, with its own lower and upper bounds for each nutrient.

In this way, an incremental diet can be defined, consisting of smaller, easier intermediate nutritional goals to reach the final desired nutritional state. One way to do this is to define a straight line from the individual's current nutritional state to the desired nutritional state and to define a sufficient number of equidistant intermediate nutritional states. Another possibility is that, in each successive intermediate state, emphasis is put on a subset of nutrients to achieve the overall desired nutritional state. In this latter case, using weighted or varying distance measures for each intermediate nutritional state may be advantageous.

This trajectory of intermediate goal states may be computed once, or it may be recomputed as each intermediate goal state is reached. In the latter case, after reaching one intermediate goal state, a new trajectory is defined as above taking into account, for example, the difficulty experienced by the individual with eating less of a certain ingredient or a certain nutrient. The new trajectory may, for instance, define smaller steps in this nutrient's dimension, or keep this nutrient's value constant for some time. Note that this difficulty can be measured by the system itself as it keeps track of the individual's edible intake. In this way, an adaptive trajectory is tailor-made with the goal of keeping the individual on track.

In one embodiment, trajectories from different users are collected, together with data about each individual user, as well as a measure of success for each trajectory. This success score can be calculated by the system by making use of, e.g., the individual's speed of making improvements or the number of adjustments that were necessary to keep the individual on track. This collection of trajectories can be analyzed to identify or generate generally successful trajectories or trajectory templates, possibly tailored to a particular type of individual user. These successful trajectories or templates could then be provided to individual users' systems improving the functionality of the system by providing validated trajectories and templates. A template describes, for example, in a more abstract way than a trajectory, a strategy to define trajectories, for instance, by first looking at daily intake of saturates and gradually introducing an increase of calcium intake.

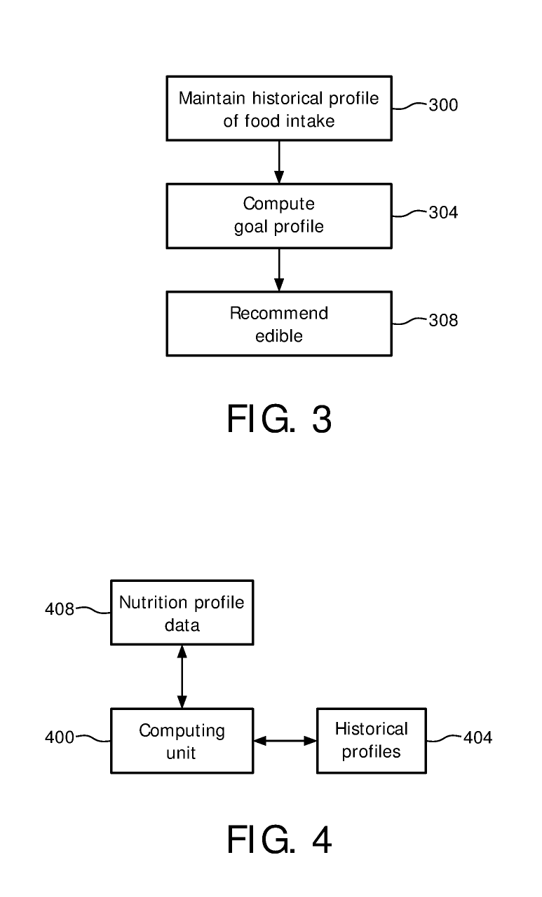

FIG. 3 presents a flowchart of a method for edible recommendation in accord with the present invention. First, a historical profile of the nutritional contributions from an individual's edible intake is generated using a historical profile data source and a nutrition profile data source (Step 300). Next, a goal nutritional intake profile is computed between the historical profile of nutritional contributions and a desired nutritional intake profile for the individual using an appropriately configured computing unit (Step 304). Then an edible (i.e., a food item, a combination of food items, a recipe, etc.) is recommended for consumption by the individual by an appropriately configured computing unit, the edible having nutritional content that moves the individual's historical profile of nutritional contributions closer to the goal nutritional intake profile (Step 308).

In one embodiment, the individual's historical profile (Step 300) is implemented using an electronic log of edible intake. The individual may manually enter an edible that he has consumed using a keyboard (physical or on-screen) or a speech interface. In other embodiments, the individual may capture an image of an edible that they have eaten using, e.g., a camera, and embodiments of the inventive system automatically identify the edible through image recognition, a scan of a barcode on the edible, some combination thereof, etc.

Alternatively, an edible browser or selection history can be used to approximate the individual's eating history (Step 300), permitting the individual to indicate which edibles they have cooked and/or consumed and in which quantity. In still other embodiments, a record of the individual's edible purchasing history can be used to approximate the individual's historical profile of edible intake (Step 300).

Each entered edible can be vectorised by consulting a nutrition profile data source such as a database for the nutritional content associated with that edible, and recast as a vector. The user's historical profile of nutritional contributions (Step 300) can then be modelled as a vector h=(h₁, h₂, . . . hₘ)=c(e₁, e₂, . . . ) that is, e.g., a vector operation such as a simple average, a moving average, an exponential average that weights recent edibles heavier, etc., of the nutritional contribution vectors associated with the edibles e₁, e₂, etc., consumed over a past time period, e.g., the past day, the past three days, the past hour, etc.

The desired nutritional intake profile may be manually specified by the individual or by a third party, such as a nurse, a dietician, or a physician. The desired nutritional intake profile may also be determined programatically by an appropriately configured computing unit if a goal nutritional state is specified.

In some embodiments, the goal nutritional profile may be the same as the desired nutritional intake profile (Step 304). In other embodiments, as discussed above, if the distance between the individual's historical nutritional profile of edible intake and the desired nutritional profile is too large, then one or more intermediate goal nutritional profiles between the historical nutritional profile and the desired nutritional profile may be computed by an appropriately configured computing unit, and embodiments of the present invention may proceed to recommend at least one edible for consumption by the individual that reduces the distance between the individual's current nutritional state, as exemplified by the individual's historical nutritional profile of edible intake, and the intermediate goal nutritional profile. As discussed above, the intermediate goal nutritional profile may be equidistant between the historical nutritional profile and the desired nutritional profile, or one of a plurality of goal nutritional profiles equidistant between the historical nutritional profile and the desired nutritional profile.

An edible e that decreases the distance d between the nutritional content of the individual's eating history h and the nutritional content of the desired goal profile g (Step 308) can be selected from a set of edibles e,e′,e″ . . . by an appropriately configured computing unit computing an updated eating history u=c(h, e) from the individual's eating history h and each of the edibles e from the set of eligible edibles and then determining whether the distance between the goal nutritional state and the nutritional content of the proposed eating history is less than the distance between the goal nutritional state and the nutritional content of the user's original history, i.e., d(c(h, e), g)<d (h, g). Edibles e that reduce that distance are eligible for recommendation (Step 308).

The set of edibles e, e′,e″ . . . eligible for recommendation may be further restricted to account for individual preferences. This is believed to make it easier for the individual to comply with long-term weight management plans. Such individual preferences may include a user-specified preference for certain tastes (e.g., “sweet” or “savory”) or flavors (e.g., “chocolate” or “strawberry”) or, conversely, a user-specified blacklist of, e.g., disfavored ingredients (e.g., “sweetbreads” or “mushrooms”) that can be used to eliminate recipes that the user will find unpalatable. One or more of these individual preferences may be disregarded in the event that they reduce the set of eligible edibles to the point that it is impractical to make recommendations.

As mentioned above, the combination function c( ) may take a variety of forms in various embodiments of the invention. The combination function may be vector operations such as an updated running average, exponential smoothing, or any other suitable method.

The updated eating history u can be the result of a running average by keeping a record of the edibles consumed by the individual for a given period p, which may be one day, two days, etc. Each edible can be expressed as a vector of nutritional content as discussed above by consulting a database of nutritional content vectors and retrieving a vector associated with the edible or consulting another data source for the nutritional content and recasting the data as vectors. The nutritional content of an eating history h can then be computed by simply adding the nutritional content vectors associated with the edibles for the period of interest. The updated eating history u can be computed by adding nutritional content vector for the current edible e to h. Hence, edibles consumed by the individual that fall outside the period p are not included in the eating history.

The nutritional content of the updated eating history u can be the result of exponential smoothing by keeping a record of the edibles consumed by the individual for a given period p, which may be one day, two days, etc. Each edible can be expressed as a vector of nutritional content as discussed above by consulting a database of nutritional content vectors and retrieving a nutritional content vector associated with the edible. The updated eating history u can then be computed by:

u=αe+(1−α)h

The operation is recursive, i.e., such that:

h=αeᵢ₋₁+(1−α)hᵢ₋₁

for edibles within the period of interest, and αϵ(0,1), with the values of the a vector being selected to weight the importance of consuming a particular nutrient. Unimportant nutrients may receive a small value of αᵢ and, conversely, important nutrients may be assigned a large αᵢ. Typically, h is set to a particular value initially, say to h₀, and then updated using the formula. This makes the maintenance of h inherently recursive and it is not recalculated every time from scratch.

The “distance” between the nutritional content of a user's eating history h, u and the goal nutritional content profile g can be measured in different ways. For each integer pϵ{1, 2, . . . }, we can define a distance measure given by:

$${d}_{p} ( h , g ) = {( {\sum }_{i = 1}^{m} {\vert {h}_{i} - {g}_{i} \vert}^{p} )}^{\frac{1}{p}}$$

For p=2, we obtain the well-known Euclidean distance that can be rewritten as:

d_{eucl}(h,g)=\sqrt{(h₁−g₁)²+ . . . +(hₘ−gₘ)²}

For p=1, we obtain the so-called Manhattan distance that can be rewritten as:

d₁(h,g)=|h₁−g₁|+ . . . +|hₘ−gₘ|

Depending on a user's medical condition, more or less weight can be attributed to each of the nutrients in the distance measure, resulting in a weighted distance measure. The weighted versions of the Euclidean and Manhattan distances are given by:

d_{eucl,w}(h,g)=\sqrt{β₁(h₁−g₁)²+ . . . +βₘ(hₘ−gₘ)²}

and

d_{1,w}(h,g)=β₁|h₁−g₁|+ . . . +βₘ|hₘ−gₘ|

respectively.

In the event that the goal nutritional profile g is a hypercube, i.e., because the desired value of one or more nutrients in the goal state is a range, then the distance between the nutritional content of the user's historical profile of edible intake h, u and the hypercube g can be defined by the minimum distance between h, u and any vector that terminates inside hypercube g. This can be expressed as:

d_{eucl}(h,g)=\sqrt{(c₁)²+ . . . +(cₘ)²}

where the contribution cᵢ for nutrient i is given by:

cᵢ=min(|hᵢ−lᵢ|,|hᵢ−uᵢ|)

when hᵢ<lᵢ or hᵢ>uᵢ and cᵢ=0 if lᵢ≤hᵢ<uᵢ. This expression can be similarly adapted for other distance measures.

Some embodiments of the present invention include functionality that, for each edible recommendation, computes and/or displays the individual's progress towards the goal nutritional state by following that particular recommendation. The visualization may take the form of, e.g., a 5-star scale in which always choosing 5-star recipes allows the user to reach the desired nutritional state within a week, while consistently choosing 1-star recipes may actually prevent the user from reaching the desired nutritional state. The visualization lets a user choose a recipe depending not only on individual preferences, but also with the awareness of the impact of the choice of the achievement of the user's overall nutritional goal.

Although the preceding discussion focuses on the recommendation of edibles that reduce the distance between the nutritional content of the individual's historical profile of edible intake and a desired nutritional content profile or goal nutritional content profile, in some cases embodiments of the invention may recommend edibles that increase the distance between the the nutritional content of the historical profile and the desired nutritional content profile. Such edibles may be thought of as “treats,” i.e., nominally unhealthy items like a dish of ice cream or a candy bar. It is believed that occasional treats may make it easier for an individual to comply with a long term weight loss plan.

Recommendations that increase the distance may be limited to situations where, e.g., the distance between the nutritional content of the individual's historical profile and the desired nutritional content profile is sufficiently close such that an increase in the distance will not prove meaningfully adverse to the individual's overall health.

It would be apparent to one of ordinary skill that the order of steps in the preceding discussion is not necessarily canonical. For example, one of ordinary skill would recognize that an edible can be recommended before, contemporaneously with, or after the computation of a goal nutritional content profile. Moreover, as embodiments of the invention are typically used in an iterative context, one would expect that multiple goal nutritional content profiles may be specified and/or computed and multiple edible recommendations made towards the achievement of these various goal nutritional content profiles. Still other embodiments allow for the recomputation of goal nutritional content profiles and recommended edibles in light of the user's response to a visualization of their progress toward the goal nutritional state.

FIG. 4 is a block diagram of an exemplary system for edible recommendation in accord with the present invention. In this embodiment, a computing unit 300 is in communication with a data source 404 for storing historical profiles of edible intake and a source of nutrition profile data 408.

The computing unit 400 may take a variety of forms in various embodiments. Exemplary computing units suitable for use with the present invention include desktop computers, laptop computers, virtual computers, server computers, smartphones, tablets, phablets, etc. Data sources 404, 408 may also take a variety of forms, including but not limited to structured databases (e.g., SQL databases), unstructured databases (e.g., Hadoop clusters, NoSQL databases), or other data sources running on a variety of computing units (e.g., desktop computers, laptop computers, virtual computers, server computers, smartphones, tablets, phablets, etc.). The computing units may be heterogeneous or homogeneous in various embodiments of the present invention. In some embodiments, the data source 304 may be connected over a wide-area network (WAN) to one or more third party data sources and create such nutrition profiles for edibles when needed. In some embodiments, the data source 308 may be connected over a wide-area network (WAN) to one or more third party data sources in order to assemble a historical profile of edible intake by retrieving and converting, e.g., purchases from a grocery store, dining records from a restaurant, etc.

The components of the systems may be interconnected using a variety of network technologies being heterogeneous or homogenous in various embodiments. Suitable network technologies include but are not limited to wired network connections (e.g., Ethernet, gigabit Ethernet, token ring, etc.) and wireless network connections (e.g., Bluetooth, 802.11x, 3G/4G wireless technologies, etc.).

In operation, the computing unit 400 queries the historical profile data source 304 for information concerning the historical profile of edible intake for an individual. The historical profile data source 404 may have such information because the individual may have manually entered such data into the data source, or it may have received such information directly or indirectly (i.e., through data entry or transmission) from a third party data source that includes a direct record of the user's consumption of edibles or a record that may be analyzed to derive such information (e.g., a receipt of purchases at a supermarket, an Instagram feed of pictures of consumed edibles, etc.).

The computing unit 400 queries the nutrition profile data source 408 for information concerning the nutrition content profile of at least one edible, such as a food item, a meal, a recipe, a combination of food items, etc., taken from the historical profile of edible intake or at least one edible intended for recommendation. The nutrition profile data source 408 may have such information stored locally, or it may contact other data sources to obtain the relevant nutrition information as necessary, converting it to the vector representation discussed above.

Having received the historical profile of edible intake for an individual, the computing unit 400 may compute a goal nutrition profile between the nutritional content of the historical profile and a desired nutrition profile. As discussed above, the goal nutrition profile may be the desired nutrition profile or an intermediate nutritional state therebetween, such as an intermediate nutritional state reachable by the individual in a reasonable time or with a reasonable effort.

With a computed goal nutrition profile and access to a nutritional profile data source, the computing unit 400 can recommend at least one edible for consumption by the individual that reduces the distance between the nutritional content of the historical profile and the goal nutrition profile or, in some instances, increases the distance by a nominal amount in the interest of helping the individual comply with a long-term dietary regime as discussed in greater detail above in connection with FIG. 3. The computing unit 400 may also generate a graphical display of the quality of the edible recommendation.

As discussed above, the computing unit 400 may access either data source 404, 408 first or access both data sources contemporaneously. In some embodiments, computing unit 400 is local to the individual, i.e., being located on a local area network accessed by the individual or contained in a device worn (e.g., a smartwatch) or carried (e.g., a smartphone) by the individual. In other embodiments, computing unit 400 is accessed by the individual over yet another network connection (not shown), such as a wide area network or the Internet, and the graphical presentation is delivered to the operator over such network connection. In these embodiments, the computing unit 400 includes security and web server functionality customary to such remotely-accessed devices.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

The description and illustration of one or more embodiments provided in this application are not intended to limit or restrict the scope of the present disclosure as claimed in any way. The embodiments, examples, and details provided in this application are considered sufficient to convey possession and enable others to make and use the best mode of the claimed embodiments. The claimed embodiments should not be construed as being limited to any embodiment, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the general inventive concept embodied in this application that do not depart from the broader scope of the claimed embodiments.

1. A computer-implemented method for recommending at least one edible for consumption, the method comprising:
providing a computer processor configured to:
maintain a historical profile of edible intake for an individual;
compute a goal profile based at least in part on the historical profile and a desired profile for the individual; and
recommend at least one edible for consumption by the individual, wherein the recommended at least one edible is selected at least based on a history of successful recommendations to at least one other individual relating to the desired profile,
wherein each profile comprises at least one parameter, with each parameter associated with a nutrient.

2. The computer-implemented method of claim 1, the computer processor further configured to:
receive an indication of at least one edible consumed by the individual;
convert the indication of the at least one consumed edible into at least one edible consumed profile; and
update the historical profile to incorporate the at least one edible consumed profile,
wherein each edible consumed profile comprises at least one parameter, with each parameter associated with a nutrient.

3. The computer-implemented method of claim 2, wherein updating the historical profile comprises computing a weighted average of the historical profile and the at least one edible consumed profile.

4. The computer-implemented method of claim 2, wherein updating the historical profile comprises computing an unweighted average of the historical profile and the at least one edible consumed profile.

5. The computer-implemented method of claim 1, wherein at least one parameter of the goal profile is a range of values defined by a lower bound and an upper bound.

6. The computer-implemented method of claim 1, wherein the historical profile comprises a plurality of parameter sets.

7. The computer-implemented method of claim 1, wherein the recommendation applies different weights to different parameters.

8. The computer-implemented method of claim 1, wherein recommending at least one edible for consumption comprises selecting at least one edible from a database of nutritional profiles for at least one edible.

9. The computer-implemented method of claim 1, wherein the goal profile is one of a plurality of goal profiles equidistant between the historical profile and the desired profile.

10. The computer-implemented method of claim 1, wherein the goal profile is the desired profile.

11. A computer readable medium comprising computer-executable instructions for performing a method for recommending at least one edible for consumption, the medium comprising:
computer-executable instructions for maintaining a historical profile of edible intake for an individual;
computer-executable instructions for communicating at least part of the historical profile to a processing module, the processing module configured to compute a goal profile between the historical profile and a desired profile for the individual;
computer-executable instructions for receiving information from the processing module; and
computer-executable instructions for recommending at least one edible for consumption by the individual, wherein the recommended at least one edible is selected at least based on a history of successful recommendations to at least one other individual relating to the desired profile,
wherein each profile comprises at least one parameter, with each parameter associated with a nutrient.

12. The computer readable medium of claim 11, the medium further comprising computer-executable instructions for:
receiving an indication of at least one edible consumed by the individual;
converting the indication of at least one consumed edible into at least one edible consumed profile; and
updating the historical profile to incorporate the at least one edible consumed profile, wherein each edible consumed profile comprises at least one parameter, with each parameter associated with a nutrient.

13. The computer-readable medium of claim 14, wherein updating the historical profile comprises computing an average of the historical profile and the at least one edible consumed profile.

14. The computer-readable medium of claim 11, wherein the recommended edible for consumption is determined by the processing module and provided in the received information.

15. The computer readable medium of claim 11, wherein at least one parameter of the goal profile is a range of values defined by a lower bound and an upper bound.

16. The computer readable medium of claim 11, wherein the historical profile comprises a plurality of parameter sets.

17. The computer readable medium of claim 11, wherein the recommendation applies different weights to different parameters.

18. The computer readable medium of claim 11, wherein the computer-executable instructions for recommending at least one edible for consumption comprise computer-executable instructions for selecting at least one edible from a database of nutritional profiles for at least one edible.

19. The computer readable medium of claim 11, wherein the goal profile is one of a plurality of intermediate goal profiles between the historical profile and the desired profile.

20. The computer readable medium of claim 11, wherein the goal profile is the desired profile.

21. A computer readable medium comprising computer-executable instructions for performing a method for recommending at least one edible for consumption, the medium comprising:
computer-executable instructions for maintaining a historical profile of edible intake for an individual;
computer-executable instructions for computing a goal profile between the historical profile and a desired profile for the individual; and
computer-executable instructions for recommending at least one edible for consumption by the individual, wherein the recommended at least one edible is selected at least based on a history of successful recommendations to at least one other individual relating to the desired profile,
wherein each profile comprises at least one parameter, with each parameter associated with a nutrient.
